(19) United States
(12) Patent Application Publication
(10) Pub. No.: US 2003/0235850 A1
(43) Pub. Date: Dec. 25, 2003

(54) Method for in vivo identification of intracellular epitopes

(76) Inventors: Antonino Cattaneo, Trieste (IT); Michela Visintin, Trieste (IT)

Correspondence Address: Samuels, Gauthier & Stevens LLP, 225 Franklin Street, Suite 3300, Boston, MA, 02110, US

(21) Appl. No.: 10/418,388

(22) Filed: Apr. 18, 2003

Related U.S. Application Data
(63) Continuation of application No. PCT/IT01/00535, filed on Oct. 22, 2001.

(30) Foreign Application Priority Data
Oct. 24, 2000 (IT) RM2000A000561

(51) Int. Cl. C12Q 1/68; G01N 33/53; G01N 33/569; C12N 15/74

(52) U.S. Cl. 435/6; 435/7.31; 435/483

(57) ABSTRACT

A method is described for the in vivo identification of epitopes of an intracellular antigen comprising the steps of :a) co-transforming of cells by a first vector including the nucleotide sequence encoding for the region of an antibody able to recognise and bind the intracellular antigen and by a second vector comprising the nucleotide sequence encoding for a peptide; b) growing co-transformed cells in such an environment that only cells wherein the antibody region and peptide recognise and interact each other are able to replicate and/or be recognised because: the antibody region able to recognise and bind the intracellular antigen is associated with a first molecule; the peptide is associated with a second molecule; the interaction of the first with the second molecule generates a selectable phenotype and/or recognisable signal; and the interaction of the first with the second molecule occurs only when the antibody region and peptide recognise and interact each-other; c) selecting the b) cells and identify the peptide as epitope.

The present invention relates to a method for the in vivo identification of intracellular epitopes. More particularly the invention relates to a method for in vivo mapping epitopes of an antigen, which is normally present in a cell, using intracellular antibodies.

An essential element for efficiently utilize antibodies, as well as intracellular antibodies, is their epitope(s) identification, i.e. the antigen portion recognised by the antibody itself. In fact for research, diagnostic and therapy it is desirable to know the epitope of an antigen and select antibodies able to bind to the same.

Many different approaches have been used to map the epitopes of an antigen, all using in vitro technologies and very laborious procedures. The limitation of in vitro technologies derives from the fact that the antigen- antibody binding is not investigated in the natural functional context of the antigen, i.e. the intracellular environment.

A method (peptide scanning) makes use of: a) synthesis of peptides which overlap by few amino acids and, as a whole, comprise the complete amino acid sequence of the investigated antigen, and b) the challenging of each peptide with antibodies. The method is very time-consuming, laborious, expensive and, overall, not always provides satisfactory results. In fact not always an epitope recognised by an antibody consists of a linear amino acid sequence.

Presently the most useful technology for the identification of epitopes recognised by different antibodies is the use of peptides having random sequences, either selected from peptide-phage libraries or synthetically synthesized. The strategy further allows to identify peptides active as epitopes having no homology with the antigen primary sequence, thus resulting in a discontinuous or conformational epitope. The methodology, therefore, allows to map a virtual collection of epitopes recognised by a given antibody, even with no structural correlation with the starting antigen.

Alternatively mutated, truncated or deleted forms of the target antigen can be engineered, expressed in suitable cellular systems, purified and individually assayed. Obviously this procedure is very laborious too.

The authors of the present invention now provide a method which identifies in vivo, in the natural environment, epitopes of intracellular antigens by means of antibodies which are expressed and active in the cellular environment (intracellular antibodies).

The use of intracellular antibodies, i.e. antibodies or portions thereof, expressed and active within a cell, represents a successful technology to interact with the function of a protein, within its physiological intracellular environment (Cattaneo & Neuberger, 1987; Carlson, 1988; Biocca & Cattaneo, 1995; Cattaneo & Biocca, 1997; Cattaneo & Biocca, 1999; Marasco, 1997). Thus the technology allows to achieve the functional knock-out of the target protein. It is therefore possible to confer a given phenotype to a cell or organism by means of suitable intracellular antibodies.

The technology of intracellular antibodies is based on two advantageous aspects: 1) the virtually unlimited availability of the immune system (natural or artificial) repertoire, providing a source of molecules suitable for high affinity and specificity reactions against any protein, and 2) the possibility to direct a protein (and then an antibody) into different intracellular compartments by appropriated, autonomous and dominant intracellular localising signals.

The technology is validly used for research and biotechnology applications, particularly for gene therapy of human and animal pathologies, to provide experimental models of pathologies, and in plant biotechnology to produce pathogen resistant transgenic plants.

The recent big development of both human and other species genome sequencing technologies determined an increasing demand to provide technologies suitable to clarify the function of identified genes, thus resulting in a new research field, so called functional genomics. It includes methods and technologies suitable to identify gene functions in a way as possible as parallel, at high throughput and suitable to be automated. Presently this step represents a remarkable bottleneck for the industry and particularly for the procedures leading from genes to new classes of drugs.

Recently a development of the intracellular antibody technology was introduced, allowing its use as an elective methodology for programs of functional genomics: the so-called ITT (intrabody trap technology). The methodology allows to isolate or select, among all the antibodies directed against a target protein, the sub-population which is able to operate efficiently in vivo, in the intracellular environment. The technology is described in the International Patent Application PCT n. WO00/54057, herein incorporated by reference. In summary it consists of a method to select antibodies from phage-libraries or from immune splenocytes, given their ability to bind an antigen in the intracellular environment. The method allows to overcome a limitation of the intracellular antibody technology, i.e. the fact that not all of antibodies able to bind in vitro an antigen are then able to bind the same antigen when expressed as cytoplasmic antibodies. Antibodies selected by the ITT procedure, based on the two-hybrid system, are able to fold correctly in the intracellular environment and to bind the target antigen therein. Therefore they are “validated” to be used as intracellular antibodies (Visintin et al., 1999). In all of ITT's applications, the antigen is challenged with an antibody library.

The authors of the present invention advantageously adapted and modified this technology for the identification of epitopes, i.e. the antigen portions able to recognise and bind an antibody. Such epitopes, in virtue of the selection technique, are in vivo recognised by the antibody itself. According to the technology of the invention, opposite to ITT, the antibody is the constant element to be challenged with a library, or a series of antigens. This new technique of in vivo epitope mapping of a specific intracellular antibody (IVEM) represents a valid alternative to the known in vitro techniques.

The simplicity of the technique makes the same applicable for large scale screenings. The advantage of the method results also from the fact that the epitope identification occurs in vivo, without the need of peptide synthesis, but with the same if not higher accuracy than known methods. The method is able to accelerate the identification of epitopes to be useful for clinical, diagnostic and pharmacological applications, with large scale screeneings within a physiological contest.

Furthermore because it is becoming evident that active sites of a protein are more conserved than the whole structure, by means of the method of the invention, it is possible to identify ancestral proteins having similar functions.

Therefore, the method allows the in vivo identification of the epitopes of an intracellular protein. It is known that many intracellular proteins include structurally homologous domains or modules, involved in protein-protein (e.g. SH2, SH3, PH, WW, PTB, PDZ domains; Pawson, 1965) or protein-DNA interactions (e.g. zinc finger, homeo-box, helix-loop-helix, chromo domains, bromo-domains). Being able to know the antibody specificity for a domain with respect to similar domains belonging to other proteins represents an essential prerequisite of the applications designed for intracellular antibodies.

The information is essential also in all of more conventional uses of antibodies and the simplicity of the instant method provides an easy solution to this need. Accordingly an antibody of interest, targeting one of these protein modules or domains, is challenged with a library of protein domains of the same family.

Analogously the method of the invention allows to identify the specificity of a given antibody for a family of correlated proteins, as well as isoforms of a protein (i.e., isoforms resulting from alternative splicing), and the specificity of a given antibody for families of evolutionary correlated proteins. This allows phylogenetic studies to be carried out very easily.

A further very interesting application made feasible by the present invention is represented by the more easier isolation of specific antibodies for mutated forms of a given protein. In this aspect, the antigen library consists of a collection of point mutants of the antigenic protein of interest. Isolation of antibodies for mutated forms of tau intracellular proteins, present in neurodegenerative pathologies, as taupathies, (Hong, M., et al., 1998; Spillantini M G, et al., 1998; Goedert et al., 1999), or of p21-ras, present in many human cancers (Barbacid, 1987; Grand and Owen, 1991) can be carried out more easily than previously.

As exemplary experimental system the authors used tau protein, a protein involved in Alzheimer's disease. However the invention is directed to a general method for epitope identification.

It is therefore an object of the present invention a method for in vivo identification of epitopes of an intracellular antigen comprising the steps of:

a) co-transforming cells with a first vector including the nucleotide sequence encoding the region of an antibody able to recognise and bind the intracellular antigen and with a vector family, each one comprising a nucleotide sequence encoding a peptide and forming a peptide library as a whole;

b) growing co-transformed cells in such an environment that only cells wherein the antibody region and the peptide able to recognize and interact each other are able to replicate and/or be identified, given that the antibody region able to recognize and bind the intracellular antigen is associated with a first molecule; the peptide is associated with a second molecule; the interaction of the first and of the second molecule generates a selectable phenotype and/or recognisable signal; and the interaction of the first and of the second molecule occurs only when the antibody region and peptide recognize and interact each other;

c) selecting cells of b) and identify the peptide as epitope.

According to a preferred embodiment the peptide library is a library of peptide fragments of an antigen; or a library of antigens; or a series of point mutants of an antigen; or a number of phylogenetic variants of an anatigen; or a series of antigens, each comprising a variant of at least one common protein domain (as, for example, SH2, SH3, PDZ, PH, PTB, WW, SHANK, ankirine repeated domains, microtubule-binding repeated domains, ZINC-FINGER domains, omeodomains, helix-loop-helix domains) or a library of aptamer peptide fragments, also fused, with a stable framework (as for example TRXA Thioredoxin, ROP).

According to a preferred embodiment the cells of the method are yeast cells.

According to a preferred embodiment the intracellular antigen is the tau protein, or phylogenetic variants thereof, or pathology associated mutants thereof, or splicing isoforms thereof, or other proteins able to bind microtubules (as, for example, MAP proteins, namely MAP1, MAP2).

The invention is now described according to preferred embodiments, with reference to the following figures:

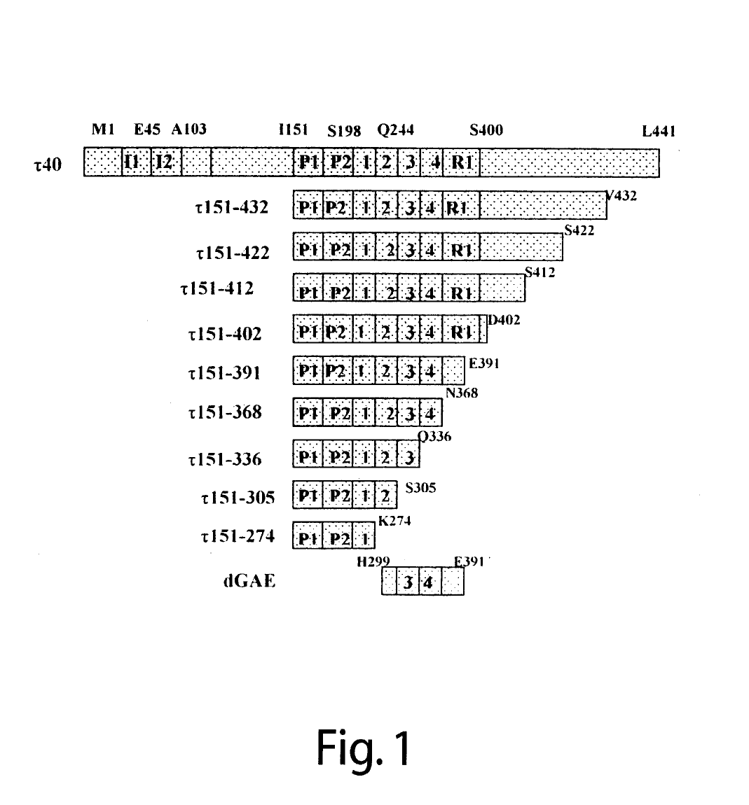

FIG. 1 is a scheme of tau protein deletion mutants;

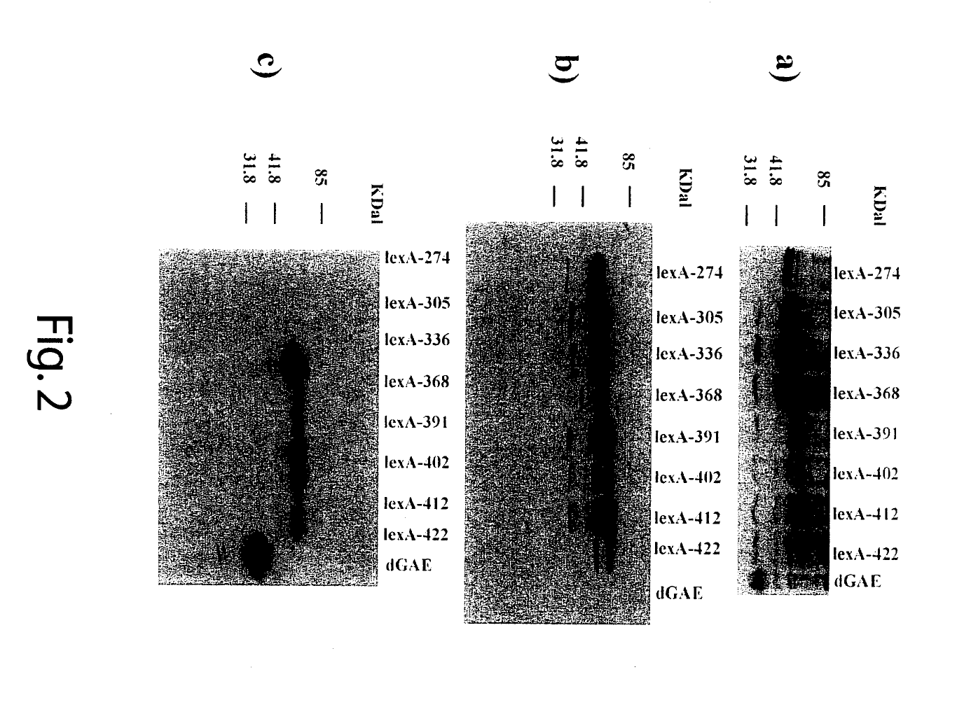

FIG. 2 shows western blot analysis of lexA-tau deletion mutant fusion proteins revealed by using a polyclonal anti-lexA antibody (panel a); a generic anti-tau monoclonal antibody (Tau-1) (panel a); another generic anti-tau monoclonal antibody (7.51) (panel c).

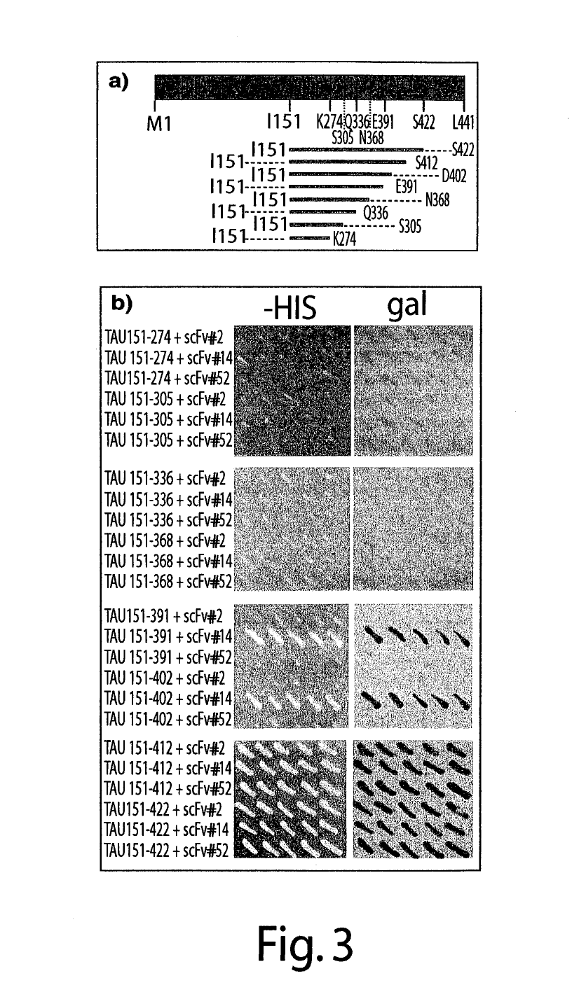

FIG. 3 shows the map of tau deletion mutants as described in Fasulo et al. 200 (panel a) and the anti-tau specificity of scFv2, scFv14 and scFv52 fragments, with respect to various deletion mutants in vivo (panel b). The epitope-scFv binding is detected by ITT technology measuring the trans-activation of HIS3 and lacZ genes and therefore the ability of the cells to grow in medium without histidine and synthesise beta-galactosidase.

### EXAMPLE 1

### Tau Mutants and Fusion Constructs with lexA

Deletion mutants of the tau protein were fused in frame to lexA binding domain. Such DNA binding domain is derived by an E. coli repressor protein (Brent & Ptashne, 1985) (Golemis & Brent, 1992) (Silver et al., 1986). LexA binding sites are located upstream to reporter genes, at the transcription activating region or promoter. The binding of lexA-tau deletion mutant proteins can reach and bind lexA binding sites but are unable to activate the transcription of reporter genes because of lacking of transcriptional activation domain. Tau fragments are illustrated in FIG. 1 and are:

Tau 151-274 including the proline rich domain and the R1 repeated sequence able to bind to microtubules;

Tau 151-305 including the proline rich domain and R1 and R2 repeated sequences able to bind to microtubules;

Tau 151-336 including the proline rich domain and R1, R2 and R3 repeated sequences able to bind to microtubules;

Tau 151-368 including the proline rich domain and R1, R2, R3 and R4 repeated sequences able to bind to microtubules;

Tau 151-391 including the proline rich domain and R1, R2, R3 and R4 repeated sequences extended up to Glu 391 residue;

Tau 151-402 including the proline rich domain and all the repeated sequences extended up to Asp 402 residue;

Tau 151-412 including the proline rich domain and all the repeated sequences extended up to Ser 412 residue;

Tau 151-422 including the proline rich domain and all the repeated sequences extended up to Ser 422 residue;

dGAE including the minimal region constituting PHF filament core observed in Alzheimer's patients.

### EXAMPLE 2

### Transformation with Tau Mutants and Expression Thereof

Plasmid DNA enconding lexA domain fused to tau protein deletion mutants is utilized to transform the L40 yeast strain (Hollenberg et al, 1995) using a standard method including lithium acetate to make permeable yeast cells. The method was provided by Clontech on the base of previously published protocols (Gietz et al., 1992; Hill et al., 1991; Schiestl & Gietz, 1989). Yeast cells are transformed using 0.1 μg of plasmid DNA and 100 μg of salmon sperm as carrier, in a solution including lithium acetate, polyethylene glycol and dimethyl sulphoxide. Transformed cells are plated on minimal medium (synthetic dropout) containing all the amino acids but tryptophan to identify the obtained phenotype. In fact the vector used to express lexA domain fused to tau deletion mutant proteins is able to complement the nutritional deficit resulting from the lack of tryptophan, thus allowing yeast growth without said amino acid.

Hybrid proteins produced by yeast cells are extracted from transformed yeast culture using an extraction buffer containing 10% β-mercaptethanol, 2% SDS, 0,1% bromophenol blue and 10% glycerol (Sambrook et al., 1990).

The expression level of fusion proteins was evaluated by western blot analysis, using anti lexa-protein polyclonal antibodies (FIG. 2a), anti Tau mAbs: Tau-1 (Roche) (FIG. 2b) and 7.51 (Novak et al., 1991) (FIG. 2c).

The experiment shows that tau deletion mutants are differently recognised by the two anti-tau antibodies, thus indicating that well expressed proteins have a suitable molecular weight and are then correctly translated and correctly express known epitopes, recognised by various used antibodies.

### Example 3

### Co-Transformation of Tau Mutants with Single Chain (ScFv) Anti-Tau Antibody Fragments, selected by Means of the ITT Method

The experiment uses a panel of single chain Fv fragments (scFv) selected by ITT technology (Visintin et al., 1999 and PCT WO00/54057). A scFv fragment library exposed on the surface of filamentous bacteriophages was screened with the purified recombinant tau protein, bound to a solid phase. After two adsorption cycles, elution, growth of eluted phages in E. coli, the “polyclonal” population of anti-tau protein enriched antibody fragments is subcloned in the two-hybrid expression vector and tested for the tau protein binding ability in two-hybrid system. From yeast cells selected in the absence of histidine, anti-tau scFv antibody fragments were isolated, able to bind tau intracellularly.

Yeast colonies, transformed with tau mutants and with each of the ITT selected scFvs, were plated on medium selective for the identification of phenotype from nutritional reporter HIS3, which is under the control of lexA binding sites, whose expression allows cells to grow in the absence of histidine.

The reconstitution of the transcriptional factor by antigen-antibody specific interaction allows the transcription of both HIS3 and lacZ reporter genes. The identification of such interaction is performed by qualitative analysis of colonies grown in the absence of histidine, and by colour change (from white to blue) of the same colony in a colorimetric assay for β-galactosidase expression.

The specificity of three different scFv (2, 14, 52) fragments against the panel of tau antibody fragments is showed in FIG. 3 and summarised in Table 1.

TABLE 1

| | Antigen | | | | | | | | |
|---|---|---|---|---|---|---|---|---|---|
| | 151-274 | 151-305 | 151-336 | 151-368 | 151-391 | 151-402 | 151-412 | 151-422 | dGAE |
| ScFv2 | − | − | − | − | − | − | + | + | − |
| ScFv14 | − | − | − | − | + | + | + | + | + |
| ScFv52 | − | − | − | − | − | − | + | + | − |

FIG. 3 shows the identification of tau protein epitopes by anti-tau single chain variable fragments, scFv2, scFv14 and scFv52. The growth on His- dishes and the β-gal phenotype shows Tau peptides interacting with one or more of the three scFV fragments. In particular S412-S422 and N368-E391 fragments do contain an in vivo recognisable epitope.

### Example 4

### Selection with the IACT Method

According to the method as disclosed in the PCT application WO 00/54057, a series of 18 new anti tau ScFvs (A to Y) is selected and characterized by means of the same panel of Tau deletion mutants of the Example 1, but dGAE.

Each antibody has been challenged in vivo with each of Tau deletion mutants, according to the method of WO 00/54057. Results are shown in Table 2 and demonstrate that the method of the invention (IVEM) allowed the identification of three main regions (1151-K 274; N 368-E 391; D 402-S 412) of Tau, recognized by antibodies selected with the IACT method.

TABLE 2

| | TAU fragments | | | | | | | |
|---|---|---|---|---|---|---|---|---|
| | I 151- | I 151- | I 151- | I 151- | I 151- | I 151- | I 151- | |
| ScFv | K 274 | S 305 | Q 336 | N 368 | E 391 | D 402 | S 412 | I 151-S 422 |
| #A | | | | | | | * | * |
| #B | | | | | | | * | * |
| #C | | | | | | | * | * |
| #D | | | | | * | * | * | * |
| #E | | | | | | | * | * |
| #F | * | * | * | * | * | * | * | * |
| #G | | | | | | | * | * |
| #K | | | | | * | * | * | * |
| #M | | | | | | | * | |
| #N | | | | | * | * | * | * |
| #O | | | | | | | * | * |
| #Q | | | | | | | * | * |
| #S | | | | | | | * | * |
| #T | | | | | | | * | * |
| #U | | | | | | | * | * |
| #V | | | | | * | * | * | * |
| #X | | | | | * | * | * | * |
| #Y | | | | | | | * | * |

Then the selection method (IVEM) allows a rapid identification of epitopes recognized in vivo by ScFvs previously selected with the IACT method, thus representing a valid alternative to traditional expensive and time consuming methods. Moreover only by means of the instant technique an easy and precise identification of the in vivo recognizing antigen region is achieved.

### BIBLIOGRAPHY

Barbacid (1987) ras genes. Annu. Rev. Biochem. 56, 779-828.

Biocca, S. & Cattaneo, A. (1995). Intracellular immunization: antibody targeting to subcellular compartments. Trends Cell Biol 5, 248-252.

Brent, R. & Pthashne, M. (1985) A eukaryotic transcriptional activator bearing the DNA specificity of a prokaryotic repressor. Cell 43 (3 Pt 2), 729-36.

Carlson, J. R. (1988). A new means of inducibly inactivating a cellular protein. Mol Cell Biol 8(6), 263846.

Cattaneo, A. & Biocca, S. (1997). Intracellular Antibodies: Development and Applications (Springer, Ed.).

Cattaneo, A. & Biocca, S. (1999). The selection of intracellular antibodies. Trends Biotechnol 17(3), 115-21.

Cattaneo, A & Neuberger, M. S. (1987). Polymeric immunoglobulin M is secreted by transfectants of non-lymphoid cells in the absence of immunoglobulin J chain. Embo J 6(9), 2753-8.

Fasulo, A. et al. (2000). J. Neurochem. 75, 624-633.

Gietz, D., et al., (1992). Improved method for high efficiency transformation of intact yeast cells. Nucleic Acids Res 20(6), 1425.

Goedert et al., (1999). Tau gene mutation in familial progressive subcortical gliosis. Nature Medicine, 5, 454-457.

Golemis, E. A. & Brent, R. (1992). Fused protein domains inhibit DNA binding by LexA. Mol Cell Biol 12(7), 3006-14

Grand and Owen (1991). The biochemistry of ras p21. Biochem. J. 279, 609-631.

Hill, J. et al., (1991). DMSO-enhanced whole cell yeast transformation [published erratum appears in Nuclei Acids Res Dec. 11, 1991; 19(23):6688] Nucleic Acids Res 19(20), 5791.

Hollenberg, S. M., et al., (1995). Identification of a new family of tissue-specific basic helix-loop-helix proteins with a two-hybrid system. Molecular and Cellular Biology 15(7), 3813-3822.

Hong, M., et al. (1998). Mutation-specific functional impairments in Distinct Tau isoforms of Heriditary FTDP-17. Science, 282,1914-1917.

Marasco, W. A. (1997). Intrabodies: turning the humoral immune system outside in for intracellular immunization. Gene Ther 4(1), 11-5.

Novak, M. et al. (1991). Difference between the tau protein of Alzheimer paired helical filament core and normal tau revealed by epitope analysis of monoclonal antibodies 423 and 7.51. Proc Natl Acad Sci USA 88(13), 5837-41.

Sambrook, J., Fritsch, E. F. & Maniatis, T (1990). Molecular Cloning: A Laboratory Manual. Cold Spring Harbor N.Y. edit (Press, C.S.H.I., Ed.)

Schiestl, R. H. & Gietz, R. D. (1989). High efficiency transformation of intact yeast cells using single stranded nucleic acids as a carrier Curr Gen 16(5-6), 339-46.

Silver, P. A., Brent, R. & Ptashne, M. (1986). DNA binding is not sufficient for nuclear localization of regulatory proteins in Saccharomyces cerevisiae. Mol Cell Biol 6(12), 4763-6.

Spillantini, M. G. et al. (1998). Mutation in the gene in familial multiple system taupathy with presenile dementia. Proc. Natl. Acad. Sci USA, 95, 7737-7741.

Visintin et al. (1999). Selection of antibodies for intracellular function using a two-hybrid system. Proc. Natl. Acad. Sci. USA 96, 11723-11728.

1. Method for in vivo identification of epitopes of an intracellular antigen comprising the steps of:
a) co-transforming of cells by a first vector including the nucleotide sequence encoding for the region of an antibody able to recognise and bind the intracellular antigen and by a vector family, anyone comprising a nucleotide sequence encoding for a peptide and forming a peptide library as a whole;
b) growing co-transformed cells in such an environment that only cells wherein the antibody region and peptide recognise and interact each other are able to replicate and/or be recognised because: the antibody region able to recognise and bind the intracellular antigen is associated with a first molecule; the peptide is associated with a second molecule; the interaction of the first with the second molecule generates a selectable phenotype and/or recognisable signal; and the interaction of the first with the second molecule occurs only when the antibody region and peptide recognise and interact each other;
c) selecting the b) cells and identifying the peptide as epitope.i

2. Method for in vivo identification of epitopes of an intracellular antigen according to claim 1 wherein the peptide library is a library of antigen peptide fragments; or a library of antigens; or a number of antigen punctiform mutants; or a number of antigen phylogenetic variants; or a number of antigens, anyone comprising a variant of at least one common protein or a library of aptamer peptide fragments, also fused, with a stable framework.

3. Method for in vivo identification of epitopes of an intracellular antigen according to claim 1 or 2 wherein the cells are yeast cells.

4. Method for in vivo identification of epitopes of an intracellular antigen according to anyone of preceding claims wherein the intracellular antigen is the tau protein or phylogenetic variants or pathology associated mutants or splicing isoforms thereof or other proteins able to bind microtubules.
